(19) United States
(12) Patent Application Publication
(10) Pub. No.: US 2022/0046953 A1
(43) Pub. Date: Feb. 17, 2022

(54) Modified Triglyceride Including Omega-3 Fatty Acid Residue

(71) Applicant: Cargill, Incorporated, Wayzata, MN (US)

(72) Inventors: Diliara IASSONOVA, Fort Collins, CO (US); Xiaolan LUO, Plymouth, MN (US)

(73) Assignee: Cargill, Incorporated, Wayzata, MN (US)

(21) Appl. No.: 17/312,564

(22) PCT Filed: Dec. 11, 2019
(86) PCT No.: PCT/US2019/065661

§ 371 Date: Jun. 10, 2021

Related U.S. Application Data
(60) Provisional application No. 62/779,244, filed on Dec. 13, 2018.

(51) Int. Cl. A23K 20/158; A23K 50/80; A23L 33/115

(52) U.S. Cl. CPC A23K 20/158; A23L 33/115; A23K 50/80

(57) ABSTRACT

Various embodiments disclosed relate to modified triglyceride including omega-3 fatty acid residue, structured fat blends including the same, food and fish feeds including the same, and methods of making any of the foregoing. A modified triglyceride includes an omega-3 fatty acid residue and a saturated fatty acid residue. A structured fat blend including the modified triglyceride can be a product of interesterification of a starting material triglyceride including an omega-3 fatty acid residue and a highly saturated triglyceride. When used in a fish feed, the modified triglyceride can experience less leakage in water than the starting material triglyceride including the omega-3 fatty acid residue.

This application claims the benefit of U.S. Provisional Application No. 62/779,244, filed Dec. 13, 2018, entitled Modified Triglyceride Including Omega-3 Fatty Acid Residue, which is hereby incorporated by reference in its entirety.

### BACKGROUND

The farming of fish such as salmon, tilapia, and shrimp, among other species, is a major world industry and much of the fish now consumed in the human diet is farmed rather than wild-caught. One of the dilemmas of fish farming, however, is that farmed fish, especially carnivorous farmed fish like salmon, need a feed source that is relatively rich in the nutrients that they would typically ingest in the wild, such as omega-3 fatty acids that are found in marine oils, such as in fish oils. Obtaining these nutrients from natural marine sources like bait fish (e.g., menhaden) and krill in order to feed farmed fish can exacerbate already declining wild fish populations that rely on those smaller fish species. Thus, fish feeds in which omega-3 fatty acids are derived from non-marine sources are particularly desirable. However, unless solid fish feed containing various triglycerides including omega-3 fatty acid residues is quickly consumed after placing in water, the triglycerides can leak from solid fish feed into the water, preventing the fish from ingesting the full dose or any of the triglyceride including the omega-3 fatty acid residue.

### SUMMARY OF THE INVENTION

Various embodiments of the present invention provide a modified triglyceride. The modified triglyceride includes an omega-3 fatty acid residue. The modified triglyceride also includes a saturated fatty acid residue.

Various embodiments of the present invention provide a structured fat blend. The structured fat blend includes an embodiment of the modified triglyceride. In various embodiments, the structured fat blend is an interesterified product of a starting material triglyceride including an omega-3 fatty acid residue and a highly saturated triglyceride.

Various embodiments of the present invention provide a structured fat blend including the modified triglyceride and a triglyceride including at least one saturated fatty acid residue, wherein the structured fat blend is an interesterified product of an omega-3 canola oil and palm stearin, fully hydrogenated cotton seed oil, fully hydrogenated high erucic rapeseed oil, fully hydrogenated soybean oil, or a combination thereof.

Various embodiments of the present invention provide a food including an embodiment of the modified triglyceride or an embodiment of the structured fat blend. The food can be a fish feed. Various embodiments of the present invention provide a method of forming the food including adding the modified triglyceride or the structured fat blend to an edible composition to form the food.

Various embodiments of the present invention provide a fish feed including a structured fat blend that is an interesterified product of omega-3 canola oil and palm stearin, fully hydrogenated cotton seed oil, fully hydrogenated high erucic rapeseed oil, fully hydrogenated soybean oil, or a combination thereof.

Various embodiments provide a method of making the modified triglyceride. The method includes reacting a fatty acid source including at least one of an omega-3 free fatty acid or a salt thereof and an ester of an omega-3 fatty acid with a highly saturated triglyceride to form the triglyceride including the omega-3 fatty acid residue and the saturated fatty acid residue. The reaction can be an interesterification, such as a chemical interesterification or an enzymatic interesterification.

Various embodiments provide a method of making a fish feed, including reacting a fatty acid source including at least one of an omega-3 free fatty acid or a salt thereof and an ester of an omega-3 fatty acid with a highly saturated triglyceride to form the triglyceride including the omega-3 fatty acid residue and the saturated fatty acid residue, and further including adding the triglyceride including the omega-3 fatty acid residue and the saturated fatty acid residue to an edible composition to form the fish feed.

Various embodiments of the modified triglycerides of the present invention that include omega-3 fatty acid residues, structured fat blends including the same, foods or fish feeds including the same, and methods of making any of the foregoing, have certain advantages over other triglycerides, blends, foods or fish feeds, and methods, at least some of which are unexpected. For example, in various embodiments, the modified triglyceride of the present invention or a structured fat blend including the same can experience less leakage (e.g., a lower rate of leakage, or zero leakage) from a food or fish food when placed in water as compared other triglycerides including omega-3 fatty acid residues. In various embodiments, the food or fish feed of the present invention including the modified triglyceride or including the structured fat blend including the same has better nutritional content as compared to other foods and fish feeds, such as due to higher omega-3 fatty acid content, and such as due to less leakage of the modified triglyceride including the omega-3 fatty acid residue.

In various embodiments, the modified triglyceride of the present invention including omega-3 fatty acid residues can be derived from sources other than palm oil, or other than animal fat, as contrasted with some other modified triglycerides that are derived from palm oil sources (e.g., palm oil derivatives, such as palm stearin, fully hydrogenated palm oil, palm olein, or palm kernel), or animal fat sources. In various embodiments, by being derived from sources other than palm oil, or other than animal fats, the modified triglyceride of the present invention can satisfy regulatory requirements in various countries.

In various embodiments, the structured fat blend of the present invention including the modified triglyceride can have a lower melting point than a blend of the starting material triglyceride including the omega-3 fatty acid residue and the highly saturated triglyceride used to form the structured fat blend. In various embodiments, the structured fat blend of the present invention including the modified triglyceride can have a lower solid fat content than a blend of the starting material triglyceride including the omega-3 fatty acid residue and the highly saturated triglyceride used to form the structured fat blend.

In various embodiments, a food or fish feed including the triglyceride of the present invention including the omega-3 fatty acid residue or including a structured fat blend including the same can provide higher digestibility and better bioavailability of the omega-3 fatty acid than other sources of omega-3 fatty acid.

In various embodiments, the modified triglyceride of the present invention including the omega-3 fatty acid residue can be derived from a crude triglyceride including the omega-3 fatty acid residues, avoiding a need for costly and time-consuming purification of the triglyceride. In various embodiments, the modified triglyceride of the present invention or the structured fat blend including the same derived from a crude triglyceride including the omega-3 fatty acid residue can have advantageous properties over the same modified triglycerides made from purified sources of the omega-3 fatty acid residue, such as a higher melting point or a higher solid fat content. In various embodiments, the modified triglyceride of the present invention including the omega-3 fatty acid residue can avoid the use of omega-3 fatty acids from fish species, and can offer a sustainable way to meet the expansion of industrial aquaculture.

### BRIEF DESCRIPTION OF THE FIGURES

The drawings illustrate generally, by way of example, but not by way of limitation, various embodiments of the present invention.

FIG. 1 illustrates Mettler dropping points of various interesterified and non-interesterified blends of palm stearin and refined, bleached, and deodorized (RBD) omega-3 canola oil, in accordance with various embodiments.

FIG. 2 illustrates Mettler dropping points of various interesterified and non-interesterified blends of fully hydrogenated high erucic rapeseed oil and crude omega-3 canola oil, in accordance with various embodiments.

FIG. 3 illustrates solid fat content (SFC) initial blends of palm stearin and RBD omega-3 canola oil, and interesterified products thereof, as a function of temperature, in accordance with various embodiments.

FIG. 4 illustrates solid fat content (SFC) of initial blends of palm stearin and crude omega-3 oil, and interesterified products thereof, as a function of temperature, in accordance with various embodiments.

FIG. 5 illustrates solid fat content (SFC) of initial blends of fully hydrogenated high erucic rapeseed oil and crude omega-3 canola oil, and interesterified products thereof, as a function of temperature, in accordance with various embodiments.

FIG. 6 illustrates changes in triacylglyceride (TAG) content of blends of palm stearin and crude omega-3 canola oil after interesterification, in accordance with various embodiments.

FIG. 7 illustrates changes in TAG content in blends of fully hydrogenated rapeseed oil and crude omega-3 canola oil after interesterification, in accordance with various embodiments.

### DETAILED DESCRIPTION OF THE INVENTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

As used herein, the term “polymer” refers to a molecule having at least one repeating unit and can include copolymers.

### Modified Triglyceride.

Various embodiments provide a modified triglyceride. The modified triglyceride includes an omega-3 fatty acid residue. The modified triglyceride can include one or two omega-3 fatty acid residues. The modified triglyceride also includes a saturated fatty acid residue. The modified triglyceride can include one or two saturated fatty acid residues. The modified triglyceride can include one omega-3 fatty acid residue and one saturated fatty acid residue, or one omega-3 fatty acid residue and two saturated fatty acid residues, or two omega-3 fatty acid residues and one saturated fatty acid residue. The triglyceride is “modified”, which as used herein means that it is not a natural triglyceride; rather, it is obtained via modification of another triglyceride.

The omega-3 fatty acid residue on the modified triglyceride can be any suitable omega-3 fatty acid residue, such as a (C₈-C₃₀)fatty acid residue including 1 to 8 carbon-carbon double bonds, or a fatty acid residue that has a carbon count of C₈ or more, or less than, equal to, or greater than C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, or that is C₃₀ or less, and that has 1 or more carbon-carbon double bonds, or less than, equal to, or greater than 2 carbon-carbon double bonds, 3, 4, 5, 6, 7, or 8 carbon-carbon double bonds or less. In an embodiment of the modified triglyceride having more than one omega-3 fatty acid, each omega-3 fatty acid can be independently selected. The omega-3 fatty acid residue can be alpha-linolenic acid (ALA, 18:3), eicosapentaenoic acid (EPA, 20:5), docosapentaenoic acid (DPA, 22:5), or docosahexaenoic acid (DHA, 22:6).

The saturated fatty acid residue can be any suitable fully saturated (e.g., fully hydrogenated) fatty acid residue. The saturated fatty acid residue can be a (C₅-C₃₀)fatty acid residue, or a (C₁₆-C₂₂)fatty acid residue, or the saturated fatty acid residue can have a carbon count of C₈ or more, or less than, equal to, or greater than C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, or that is C₃₀ or less. In an embodiment of the modified triglyceride having more than one saturated fatty acid residue, each saturated fatty acid residue can be independently selected.

In an embodiment of the modified triglyceride including only one omega-3 fatty acid residue and only one saturated fatty acid residue, the third fatty acid residue on the modified triglyceride can be any suitable fatty acid residue, such as a (C₅-C₃₀)fatty acid residue including 0 to 8 carbon-carbon double bonds, or a fatty acid residue that has a carbon count of C₈ or more, or less than, equal to, or greater than C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, or that is C₃₀ or less, and that has 0 or more carbon-carbon double bonds, or less than, equal to, or greater than 1 carbon-carbon double bonds, 2, 3, 4, 5, 6, 7, or 8 carbon-carbon double bonds or less.

The modified triglyceride can be substantially free of free fatty acids. For example, in an embodiment wherein the modified triglyceride is formed from an omega-3-containing free fatty acid and a triglyceride, the method of forming the modified triglyceride can include substantially removing free fatty acid byproducts of the reaction from the generated modified triglyceride.

### Structured Fat Blend.

Various embodiments of the present invention provide a structured fat blend including an embodiment of the modified triglyceride that includes an omega-3 fatty acid residue and a saturated fatty acid residue. The structured fat blend can also include another triglyceride different than the modified triglyceride, with the other triglyceride including at least one saturated fatty acid residue. The other triglyceride can include one, two, or three saturated fatty acid residues, wherein each fatty acid residue can be any suitable fully saturated (e.g., fully hydrogenated) fatty acid residue. The saturated fatty acid residue can be a (C₅-C₃₀)fatty acid residue, or a (C₁₆-C₂₂)fatty acid residue, or the saturated fatty acid residue can have a carbon count of C₈ or more, or less than, equal to, or greater than C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, or that is C₃₀ or less. In an embodiment of the other triglyceride including more than one saturated fatty acid residue, each saturated fatty acid residue can be independently selected. The blend can be a homogeneous blend.

Any suitable proportion of the structured fat blend can be the modified triglyceride. The modified triglyceride can be about 1 wt % to about 99 wt % of the blend, or about 20 wt % to about 80 wt % of the blend, or about 1 wt % or less, or less than, equal to, or greater than about 2 wt %, 3, 4, 5, 6, 8, 10, 12, 14, 16, 18, 20, 25, 30, 35, 40, 45, 50, 55, 60, 65, 70, 75, 80, 82, 84, 86, 88, 90, 91, 92, 93, 94, 95, 96, 97, 98, or about 99 wt % or more.

Any suitable proportion of the structure fat blend can be the other triglyceride including at least one saturated fatty acid residue. The triglyceride including at least one saturated fatty acid residue can be about 1 wt % to about 99 wt % of the blend, or about 20 wt % to about 80 wt % of the blend, or about 0 wt %, or about 1 wt % or less, or less than, equal to, or greater than about 2 wt %, 3, 4, 5, 6, 8, 10, 12, 14, 16, 18, 20, 25, 30, 35, 40, 45, 50, 55, 60, 65, 70, 75, 80, 82, 84, 86, 88, 90, 91, 92, 93, 94, 95, 96, 97, 98, or about 99 wt % or more.

The blend can be an interesterified product of a starting material triglyceride including an omega-3 fatty acid residue and a highly saturated triglyceride, such as an interesterified product of an omega-3 canola oil and palm stearin, fully hydrogenated cotton seed oil, fully hydrogenated high erucic rapeseed oil, fully hydrogenated soybean oil, or a combination thereof. The structured fat blend can have any suitable melting point relative to a blend of a starting material triglyceride including the omega-3 fatty acid residue and a highly saturated triglyceride used to form the structured fat blend, such as a higher or lower melting point; in some embodiments, the melting point is lower. The structured fat blend can have any suitable solid fat content (SFC) as compared to a blend of a starting material triglyceride including the omega-3 fatty acid residue and a highly saturated triglyceride used to form the structured fat blend, such as a higher or lower SFC; in some embodiments, the SFC content is lower.

The structured fat blend, as compared to a blend of a starting material triglyceride including the omega-3 fatty acid residue and a highly saturated triglyceride used to form the structured fat blend, can have any suitable relative amount of trisaturated (S3) triglyceride, such as about 55% less to about 1 wt % less S3 triglyceride content, or about 2 wt % less to about 35 wt % less, or about 2% less to about 4% less, or about 8% less to about 20% less, or about 55 wt % less, or less than, equal to, or greater than about 50 wt % less, 45, 40, 35, 30, 25, 20, 18, 16, 14, 12, 10, 8, 6, 4, 2, or about 1 wt % less. The structured fat blend can have any suitable amount of S3 triglycerides, such as about 5 wt % to about 95 wt % of the structured fat blend, about 10 wt % to about 90 wt %, about 1 wt % to about 70 wt %, or about 6 wt % to about 50 wt %, or about 7 wt % to about 19 wt %, or about 1 wt % or less, or less than, equal to, or greater than about 2 wt %, 4, 6, 8, 10, 12, 14, 16, 18, 20, 25, 30, 35, 40, 45, 50, 55, 60, 65, 70, 75, 80, 85, 90 wt %, or about 95 wt % or more.

The structured fat blend, as compared to a blend of a starting material triglyceride including the omega-3 fatty acid residue and a highly saturated triglyceride used to form the structured fat blend, can have any suitable relative amount of disaturated-monounsaturated (S2U) triglyceride content, such as about 20 wt % less to about 70 wt % more S2U triglyceride content, or about 1 wt % more to about 50 wt % more, or about 1 wt % more to about 5 wt % more, or about 20 wt % more to about 50 wt % more, or about 20 wt % less, or less than, equal to, or greater than about 15 wt % less, 10, 5, 2, 1 wt % less, 0 wt %, 1 wt % more, 2 wt %, 3, 4, 5, 6, 8, 10, 12, 14, 16, 18, 20, 25, 30, 35, 40, 45 wt %, or about 50 wt % more. The structured fat blend can have any suitable amount of S2U triglycerides, such as about 1 wt % to about 70 wt % of the structured fat blend, about 19 wt % to about 51 wt %, or about 19 wt % to about 40 wt %, or about 23 wt % to about 51 wt %, or about 1 wt % or less, or less than, equal to, or greater than about 2 wt %, 4, 6, 8, 10, 12, 14, 16, 18, 20, 22, 24, 26, 28, 30, 32, 34, 36, 38, 40, 42, 44, 46, 48, 50, 55, 60, 65 wt %, or about 70 wt % or more.

The structured fat blend, as compared to a blend of a starting material triglyceride including the omega-3 fatty acid residue and a highly saturated triglyceride used to form the structured fat blend, can have any suitable relative amount of monsaturated-diunsaturated (SU2) triglyceride content, such as about 25 wt % less to about 70 wt % more, about 25 wt % less to about 40 wt % more SU2 triglyceride content, or about 5% less to about 20 wt % more, or about 7 wt % more to about 11 wt % more, or about 5 wt % less to about 20 wt % more, or about 25 wt % less, or less than, equal to, or greater than about 15 wt % less, 10, 8, 6, 4, 2, 1 wt % less, 0 wt %, 1 wt % more, 2, 4, 6, 7, 8, 9, 10, 11, 12, 14, 16, 18, 20, 25, 30, 35, 40, 45, 50, 55, 60, 65 wt %, or about 70 wt % more. The structured fat blend can have any suitable amount of SU2 triglycerides, such as about 1 wt % to about 70 wt % of the structured fat blend, about 5 wt % to about 27 wt %, about 29 wt % to about 35 wt %, about 5 wt % to about 47 wt %, or about 1 wt % or less, or less than, equal to, or greater than about 2 wt %, 4, 6, 8, 10, 12, 14, 16, 18, 20, 22, 24, 26, 28, 30, 32, 34, 36, 38, 40, 42, 44, 46, 48, 50, 55, 60, 65 wt %, or about 70 wt % or more.

The structured fat blend, as compared to a blend of a starting material triglyceride including the omega-3 fatty acid residue and a highly saturated triglyceride used to form the structured fat blend, can have any suitable relative amount of triunsaturated (U3) triglyceride content, such as about 55 wt % less to about 1 wt % less U3 triglyceride content, such as about 2 wt % less to about 25 wt % less, or about 6 wt % less to about 11 wt % less, or about 19 wt % less to about 35 wt % less, or about 55 wt % less, or less than, equal to, or greater than about 50 wt % less, 45, 40, 35, 32, 30, 28, 26, 24, 22, 20, 18, 16, 14, 12, 11, 10, 9, 8, 7, 6, 5, 4, 2 wt %, or about 1 wt % less. The structured fat blend can have any suitable amount of U3 triglycerides, such as about 1 wt % to about 60 wt % of the structured fat blend, or about 1 wt % to about 39 wt %, or about 12 wt % to about 38 wt %, or about 1 wt % to about 17 wt %, or about 1 wt % or less, or less than, equal to, or greater than about 2 wt %, 4, 6, 8, 10, 12, 14, 16, 18, 20, 22, 24, 26, 28, 30, 32, 34, 36, 38, 40, 45, 50, 55 wt %, or about 60 wt % or more.

### Method of Making the Modified Triglyceride or a Structured Fat Blend Including the Same.

Various embodiments of the present invention provide a method of making the modified triglyceride that includes an omega-3 fatty acid residue, or the structured fat blend including the modified triglyceride and the triglyceride including at least one saturated fatty acid residue. The method can be any suitable method that forms the modified triglyceride of the structured fat blend including the same. The method can include reacting a fatty acid source including at least one of an omega-3 free fatty acid or a salt thereof and an ester of an omega-3 fatty acid with a highly saturated triglyceride to form the triglyceride including the omega-3 fatty acid residue and the saturated fatty acid residue.

The highly saturated triglyceride can be any suitable highly saturated triglyceride that forms the modified triglyceride or structured fat blend that includes the same. The highly saturated triglyceride can be a fully saturated, or fully hydrogenated, triglyceride. The highly saturated triglyceride can be any suitable triglyceride including fully saturated fatty acid residues, wherein the fatty acid residues can be independently chosen from a (C₈-C₃₀)fatty acid residue, or a (C₁₆-C₂₂)fatty acid residue, or the saturated fatty acid residue can have a carbon count of C₈ or more, or less than, equal to, or greater than C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, or that is C₃₀ or less. The highly saturated triglyceride can be provided by palm stearin, fully hydrogenated cotton seed oil, fully hydrogenated high erucic rapeseed oil, fully hydrogenated soybean oil, or a combination thereof. The highly saturated triglyceride can be a purified triglycerides (e.g., deodorized, bleached, refined) or a crude triglyceride (e.g., not subjected to deodorization, bleaching, or refinement).

In some embodiments, the fatty acid source includes an omega-3 free fatty acid. In such an embodiment, during the chemical reaction between the fatty acid source and the highly saturated triglyceride, one or more of the saturated fatty acid residues on the highly saturated triglyceride is replaced with an omega-3 fatty acid residue corresponding to the omega-3 free fatty acid starting material, or one of more of the fatty acid residues on the fatty acid source is replaces with a saturated fatty acid residue from the highly saturated triglyceride. The method can include separating a non-omega 3-containing free fatty acid product of the reaction from the triglyceride including the omega-3 fatty acid residue and the saturated fatty acid residue from the highly saturated triglyceride, such that the modified triglyceride produced or structured fat blend including the same is substantially free of free fatty acids.

In some embodiments, the reaction can be an interesterification, wherein one or more fatty acid residues on the fatty acid source are rearranged with fatty acid residues on the highly saturated triglyceride. In such an embodiment, the fatty acid source is an ester of the omega-3 fatty acid, such as a triglyceride including an omega-3 fatty acid residue. The interesterification can be a chemical interesterification. The chemical interesterification can be performed in the presence of any suitable interesterification catalyst, such as an alkoxide salt, such as sodium methoxide. In other embodiments, the esterification can be an enzymatic interesterification, which can produce a different distribution of triacylglycerides in the modified triglyceride or structured fat blend including the same than a chemical interesterification. During an enzymatic interesterification, one or more suitable enzymes are used to catalyze the reaction, such as Lipozyme TL IM (EC 3.1.1.3), a silica-granulated Thermomyces lanuginosa, which is sn-1,3 specific, provided by Novo Nordisk A/S (Bagsvaerd, Denmark); Lipozyme 435, a recombinant lipase from Candida antarctica, immobilized on a lewatit VP OC 1600; Novozyme 435, lipase B from C. antarctica, immobilized on a macroporous acrylic resin, sn-1,3-specific or nonspecific depending on the substrates, 10,000 PLU/g; Lipozyme RM IM, lipase from Rhizomucor miehei, immobilized on a macroporous anion exchange resin, sn-1,3-specific, 275 IUN/g; or a commercial food-grade lipase from Rhizopus oryzae (L036P, Biocatalysts, Cardiff, UK) immobilized on polysiloxane-polyvinyl alcohol (SiO₂-PVA) composite.

The ester of the omega-3 fatty acid used in the interesterification can be any suitable ester, such as a (C₁-C₂₀)hydrocarbyl ester, or a (C₁-C₅)alkyl ester. The ester can be a triglyceride including a residue of the omega-3 fatty acid, such as any suitable triglyceride omega-3 fatty acid source, such as any suitable oil or fat, such as algal oil, omega-3 canola oil (e.g., canola oil including omega-3 fatty acid residues), an other plant-based oil including omega-3 fatty acid residues, any suitable marine-based (e.g., fish oil) or non-marine-based oil, or a combination thereof. The triglyceride can be a triglyceride formed from a plant without genetic modification, or the triglyceride can be formed from a plant that has been genetically modified to form a larger amount of triglycerides including omega-3 fatty acid residues. For example, the omega-3 canola oil can be sourced from a non-genetically engineered plant, or from a genetically engineered plant. The triglyceride can be a crude triglyceride, such as a triglyceride that has not been subjected to deodorization, bleaching, or refinement, which may further include one or more sterols, tocopherols, lecithins, phosphorus lipids, or a combination thereof, in any suitable concentration. In other embodiments, the triglyceride can be a purified triglyceride, such as having been subjected to deodorization, bleaching, and refinement.

### Food or Fish Feed, and Method of Forming the Same.

Various embodiments of the present invention provide a food including the modified triglyceride that includes an omega-3 fatty acid residue, or that includes the structured fat blend including the modified triglyceride. The food can be any food, such as any solid food that can have the modified triglyceride or structured fat blend including the same blended therewith. The food can be a human food or an animal food.

In some embodiments, the food is a fish feed. The fish feed can be suitable for feeding to any fish. In some examples, the fish feed is a salmon fish feed suitable for salmon aquaculture (i.e., salmon farming). The fish feed can have any suitable form, such as a pellet or a powder. In some embodiments, when placed in water the fish feed can experience less leakage of the omega-3 fatty acid residue from the fish feed than the same fish feed having the modified triglyceride replaced with the starting material triglyceride including the omega-3 fatty acid used to form the structured fat blend, or having the structured fat blend replaced with a non-interesterified blend of the starting material triglyceride including the omega-3 fatty acid used to form the structured fat blend and the highly saturated triglyceride.

A fish feed pellet can be a solid particle that may be of any size or shape suitable for use as a fish feed. Pellets are often mechanically extruded into roughly cylindrical or spherical shapes by an extruder device, but they may also be prepared as flakes or other flat shapes, for example, and their length and diameter may also vary depending upon what is desirable for storage, transport, environmental concerns, and the type of fish they are intended for feeding. Fish feeds may also be provided in powder form, e.g., as fine, small particles.

Any suitable proportion of the food or fish feed can be the modified triglyceride, or the structured fat blend, such as about 0.1 wt % to about 100 wt % of the food, or about 2.5 wt % to about 20 wt %, or about 0.1 wt % or less, or less than, equal to, or greater than 1%, 2, 3, 4, 5, 6, 8, 10, 12, 14, 16, 18, 20, 25, 30, 35, 40, 45, 50, 55, 60, 65, 70, 75, 80, 85, 90, 95, or about 99 wt % or more.

The fish feed can further include any suitable component of a fish feed. For example, the fish feed can include fish oil, fish meal, soy meal, cereals, binders such as starches, appropriate vitamins and minerals, glycerol monostearate, an oil component (e.g., soy oil, linseed oil), lecithin, or a combination thereof.

Various embodiments of the present invention provide a method of forming the food or fish feed that includes the modified triglyceride that includes an omega-3 fatty acid residue, or that includes the structured fat blend including the modified triglyceride and the triglyceride including at least one saturated fatty acid residue. The method can be any suitable method that forms the food. The method can include adding the modified triglyceride or the structured fat blend including the same to an edible composition (e.g., edible by animals, such as fish, or humans) to form the food.

### EXAMPLES

Various embodiments of the present invention can be better understood by reference to the following Examples which are offered by way of illustration. The present invention is not limited to the Examples given herein.

Materials. All oils including crude and refined, bleached, and deodorized (RBD) omega-3 canola oils, palm stearin, fully hydrogenated cotton seed oil, and fully hydrogenated high erucic rapeseed oil, were obtained from Cargill. The omega-3 canola oils were produced from plants genetically-engineered to produce a larger amount of omega-3-containing oils, as described in U.S. provisional application No. 62/657,128, filed Apr. 13, 2018. The bleaching clays were from Oil-Dri Corporation of America, and the Trisyl® silica was from W.R. Grace. Sodium methoxide powder was purchased from Sigma-Aldrich. Menhaden fish oil was obtained from Omega Protein.

Blend preparation. Oil blends were prepared by melting and mixing highly saturated oil and omega-3 canola oil in weight ratios of from 80:20 to 30:70, as shown in Table 1.

TABLE 1
Weight ratios of highly saturated oil and omega-3 canola oil in
Samples 1-6.

| Sample | 1 | 2 | 3 | 4 | 5 | 6 |
|---|---|---|---|---|---|---|
| Palm stearin/RBD omega-3 | 80/20 | 70/30 | 60/40 | 50/50 | 40/60 | 30/70 |
| canola oil | | | | | | |
| Palm stearin/crude omega-3 | 80/20 | 70/30 | 60/40 | 50/50 | 40/60 | 30/70 |
| canola oil | | | | | | |
| Fully hydrogenated cotton | 80/20 | 70/30 | 60/40 | 50/50 | 40/60 | 30/70 |
| seed oil/crude omega-3 | | | | | | |
| canola oil | | | | | | |
| Fully hydrogenated high | 80/20 | 70/30 | 60/40 | 50/50 | 40/60 | 30/70 |
| erucic rapeseed oil/crude | | | | | | |
| omega-3 canola oil | | | | | | |

### Example 1. Chemical Interesterification of Samples 1-6

Chemical interesterification. 100 g of blends were added to a 500 mL round bottom flask and dried under vacuum at 95° C. for 30 min. Sodium methoxide (0.2-0.6%, w/w) was then added to the flask as a catalyst. After 30 min of stirring at 700 rpm at 95° C. under vacuum, water was added to the flask to deactivate catalyst and terminated reaction. Subsequently, the interesterified mixture was separated in a jacketed separatory funnel and washed several times with hot water (about 70° C.) to remove deactivated catalyst derivatives and soaps formed by the reaction between sodium ion and free fatty acids present in the reaction. After drying under vacuum, the interesterified mixture was bleached with 1% bleaching clay and 1% Trisyl® silica at 95° C. under vacuum for 30 min Finally, the chemical interesterified products were obtained after filtration.

### Example 2. Mettler Dropping Point (MDP)

The Mettler dropping point of a sample is the temperature at which the first drop falls or flows out of the opening of a standardized sample cup. It was determined according to American Oil Chemists' Society (AOCS) method Cc 18-80.

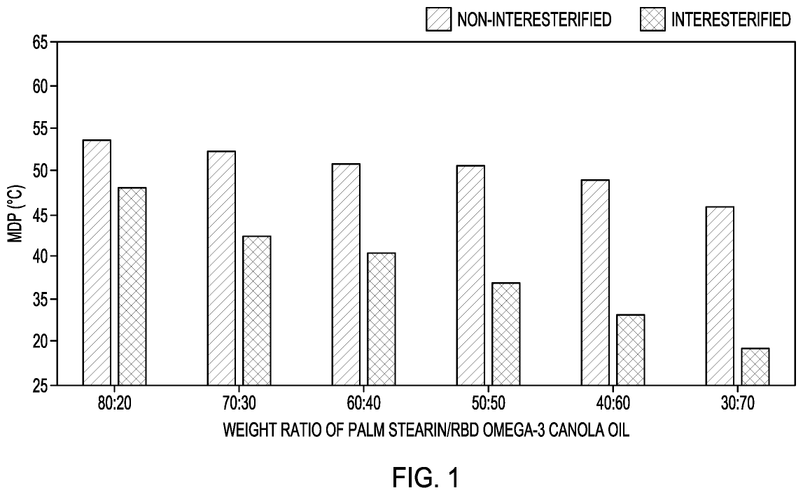

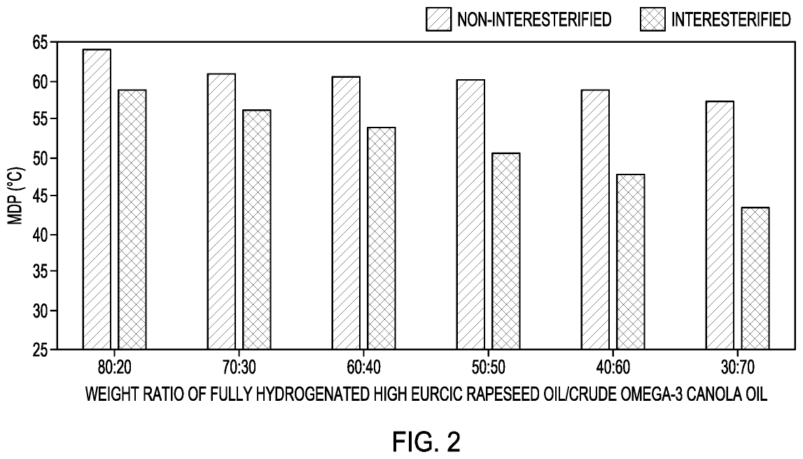

The MDPs of interesterified and non-interesterified blends of palm stearin and RBD omega-3 canola oil, and of fully hydrogenated high erucic rapeseed oil and crude omega-3 canola oil, are shown in FIGS. 1-2. The interesterified products had decreased MDPs compared to non-interesterified blends at all ratios. This can be explained by the decrease of higher-melting trisaturated triglycerides after interesterification. With an increase of content of palm stearin or fully hydrogenated oil in the blends, the MDP of the interesterified products increased. At the same weight ratio of oil blends, interesterified products of fully hydrogenated high erucic rapeseed oil and crude omega-3 canola oil demonstrated higher MDP than interesterified palm stearin and crude omega-3 canola oil. This indicated that the more saturated fat in the blend, the higher the MDP of the interesterified products. Compared to interesterified products with RBD omega-3 canola oil (e.g., interesterified palm stearin and RBD omega-3 canola oil, MDP ranged from 29.3° C. to 48° C. as shown in FIG. 1), interesterified products containing crude omega-3 canola oil showed increased MDP (e.g., interesterified palm stearin and crude omega-3 canola oil, MDP ranged from 37.7° C. to 48.2° C.). This may be attributed to the presence of lecithin, phosphorus lipids, and sterols.

### Example 3. Solid Fat Content (SFC)

Solid fat content in samples at temperatures of 10° C., 15, 20, 25, 30, 35, 40, 45, and 50° C., was determined using Nuclear Magnetic Resonance (NMR) spectrometry according to AOCS method Cd 16b-93.

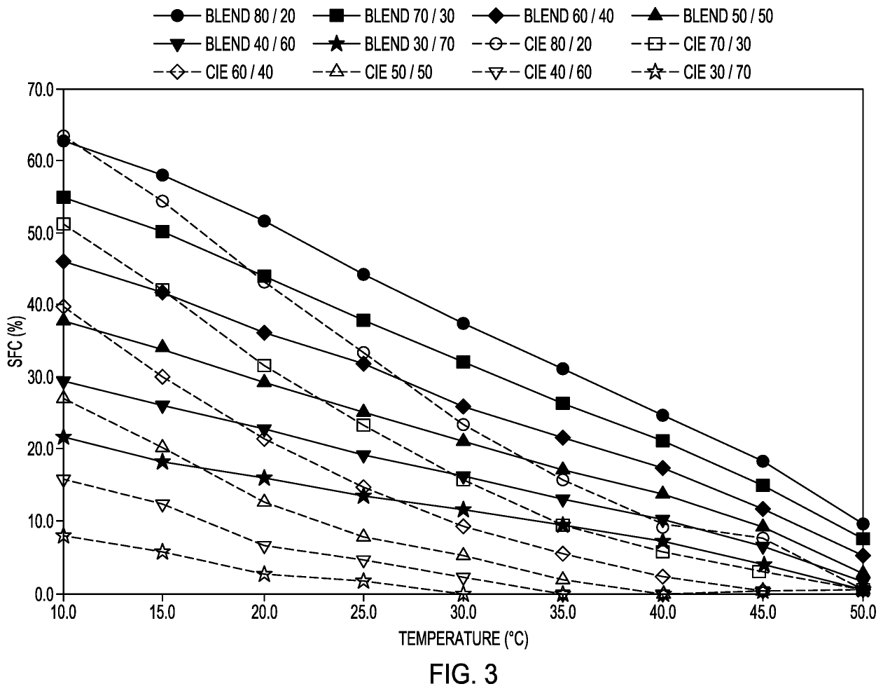

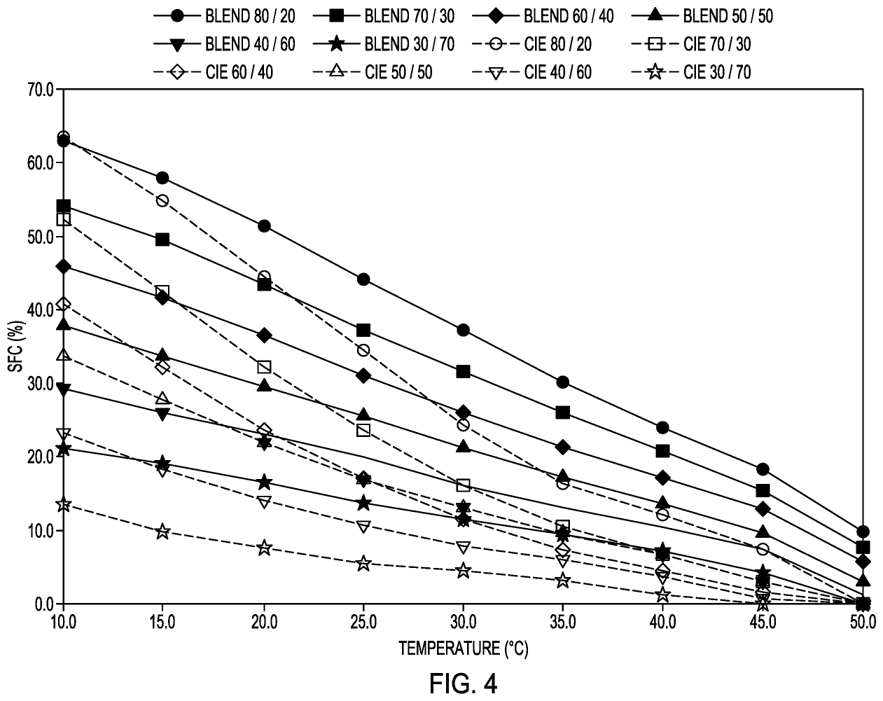

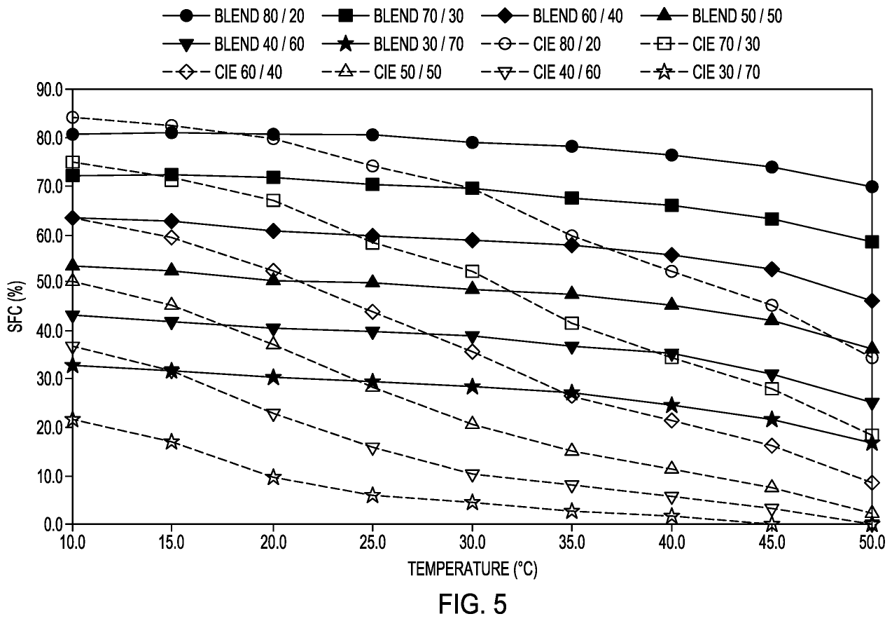

FIGS. 3-5 illustrate SFC curves of interesterified products and their blends as a function of temperature. FIG. 3 shows solid fat content (SFC) of interesterified products and initial blends of palm stearin and RBD omega-3 canola oil as a function of temperature. FIG. 4 shows solid fat content (SFC) of interesterified products and initial blends of palm stearin and crude omega-3 oil as a function of temperature. FIG. 5 shows solid fat content (SFC) of interesterified products and initial blends of fully hydrogenated high erucic rapeseed oil and crude omega-3 canola oil as a function of temperature. It was observed that interesterification tended to result in decreased SFC for all blends at all temperatures. Generally, at a lower temperature (e.g., 10° C. and 15° C.), large changes in SFC were observed for all the blends with 30-40% hard fat such as palm stearin and fully hydrogenated oil. At a higher temperature (35° C.), as shown in FIGS. 3-4, the largest changes in SFC for the blends with 70-80% of palm stearin were seen. SFC changes in chemical interesterification (CIE) products of palm stearin and RBD omega-3 canola oil were similar to those in CIE products of palm stearin and crude omega-3 canola oil. However, the latter had slightly higher SFC than the former likely due to the impact from lecithin, phosphorus lipids, and sterols present in crude omega-3 canola oil. This agreed with the changes in MDP of these CIE products. The use of crude omega-3 canola oil increased MDP and SFC of the interesterified blends. Compared to CIE products of palm stearin and crude omega-3 canola oil, CIE products with fully hydrogenated oil had higher SFC because their saturated fat content was higher than those from palm stearin. As the saturated fat content in blends increased, SFC of CIE products increased.

### Example 4. Triacylglyceride (TAG) Profile Analysis

The TAG profile of the blends and their interesterified products were analyzed using high-performance liquid chromatography-mass spectrometry (HPLC-MS).

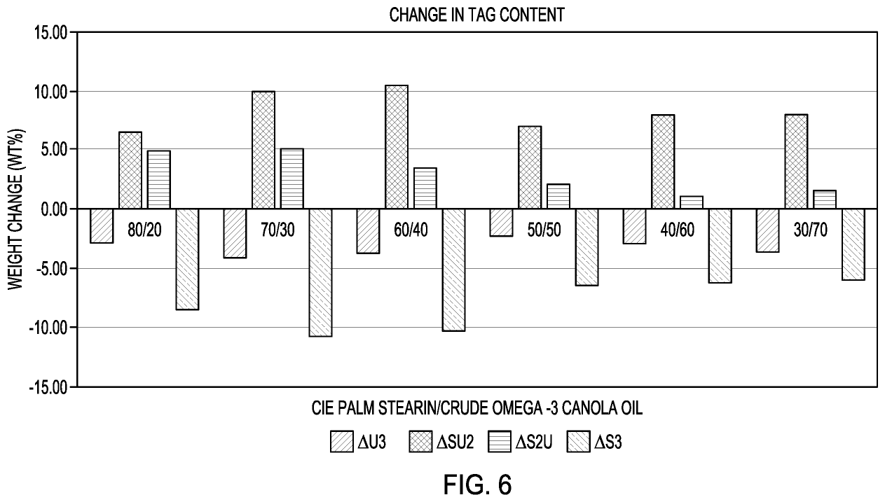

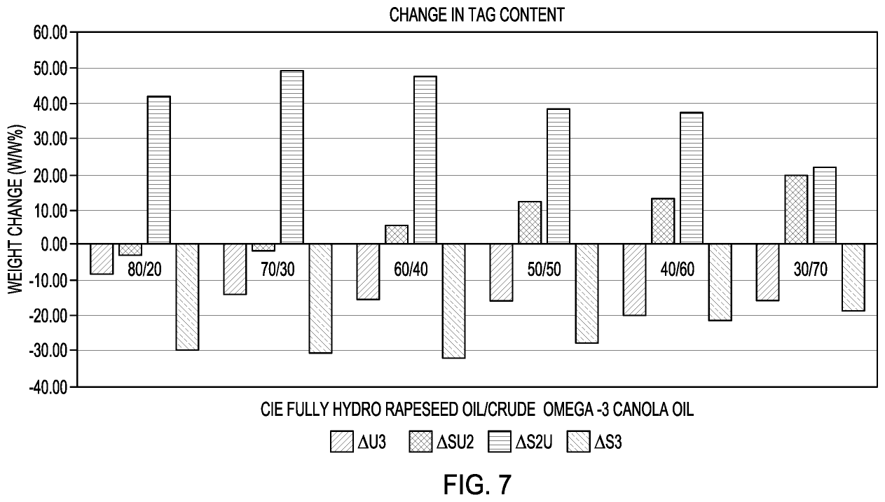

Chemical interesterification is a process in which fatty acids are randomly distributed through three positions of glycerol backbone. The TAG composition of initial blends and interesterified products is summarized in Tables 2 and 3. After interesterification, a substantial decrease in trisaturated (S3) and triunsaturated (U3) TAG content was observed for all blends, and more disaturated-monounsaturated (S2U) and monsaturated-diunsaturated (SU2) TAGs formed. Additionally, blends containing fully hydrogenated oil showed more significant changes in S3, S2U, SU2, and U3 TAGs than blends with palm stearin. FIG. 6 shows changes in TAG content of in blends of palm stearin and crude omega-3 canola oil after interesterification. In CIE products of palm stearin and crude omega-3 canola oil, S3 content decreased by 6-11%, U2 content decreased by less than 5%, while SU2 content increased by 7-11% and S2U slightly increased (less than 5%), as shown in FIG. 6. In contrast, in CIE products of fully hydrogenated rapeseed oil and omega-3 canola oil, S3 content decreased by 19-32% and U3 content decreased by 8-21%, while S2U increased by 22-50% and SU2 content increased up to 20%, as shown in FIG. 7, which shows changes in TAG content of in blends of palm stearin and crude omega-3 canola oil after interesterification

TABLE 2
Triglyceride composition of blends of palm stearin and crude omega-
3 canola oil before (B) and after (I) interesterification.

| Blends (palm | | | | | | | | |
|---|---|---|---|---|---|---|---|---|
| stearin/crude | Triglyceride (wt %) | | | | | | | |
| omega-3 canola | S3 | | S2U | | SU2 | | U3 | |
| oil, w/w) | B | I | B | I | B | I | B | I |
| 80/20 | 26.94 | 18.26 | 34.67 | 39.54 | 23.05 | 29.62 | 15.35 | 12.58 |
| 70/30 | 24.53 | 13.66 | 30.81 | 35.92 | 24.12 | 34.03 | 20.54 | 16.38 |
| 60/40 | 22.15 | 11.75 | 27.72 | 31.27 | 24.85 | 35.39 | 25.28 | 21.59 |
| 50/50 | 19.86 | 13.06 | 24.67 | 26.74 | 25.81 | 32.85 | 29.66 | 27.35 |
| 40/60 | 16.57 | 10.39 | 21.04 | 22.04 | 26.19 | 34.31 | 36.20 | 33.26 |
| 30/70 | 13.60 | 7.60 | 17.83 | 19.33 | 26.95 | 35.03 | 41.62 | 38.04 |

TABLE 3
Triglyceride composition of blends of fully hydrogenated rapeseed
oil and crude omega-3 canola oil before (B) and after
(I) interesterification.

| Blends (Fully | | | | | | | | |
|---|---|---|---|---|---|---|---|---|
| hydrogenated | | | | | | | | |
| rapeseed oil/crude | Triglyceride (wt %) | | | | | | | |
| omega-3 canola | S3 | | S2U | | SU2 | | U3 | |
| oil, w/w) | B | I | B | I | B | I | B | I |
| 80/20 | 79.36 | 49.56 | 0.76 | 42.87 | 7.81 | 5.58 | 9.43 | 1.46 |
| 70/30 | 65.14 | 34.67 | 0.68 | 50.19 | 13.52 | 12.13 | 16.20 | 2.34 |
| 60/40 | 56.00 | 23.80 | 0.76 | 48.73 | 16.50 | 21.61 | 20.21 | 4.85 |
| 50/50 | 45.90 | 17.78 | 1.14 | 39.62 | 19.98 | 32.03 | 24.35 | 8.21 |
| 40/60 | 37.70 | 16.42 | 0.94 | 38.40 | 22.61 | 35.61 | 28.89 | 8.25 |
| 30/70 | 25.00 | 6.32 | 1.37 | 23.17 | 27.58 | 47.21 | 33.39 | 17.40 |

### Example 5. Anti-Seepage Test

The interesterified products were heated at 70° C. in an oven until the fat was completely melted. The melted oil was then transferred to 25 ml graduated cylinder which was preheated at 70° C. The sample cooled down at room temperature for a few days and the ultimate oil loss was recorded according to the equation Oil loss (%)=(Volume of clear oil/Volume of total oil)*100%.

The oil loss content of all interesterified products was evaluated and the results are shown in Table 4. No oil loss was observed for any CIE products with crude omega-3 canola oil and CIE products of palm stearin and RBD omega-3 canola oil at weight ratio from 80/20 to 60/40. CIE products of palm stearin and RBD omega-3 canola oil with weight ratio of 50/50 had 6% oil loss. However, as the weight ratio of palm stearin and RBD omega-3 canola oil decreased to 40/60, oil loss increased to 70%. With a further decrease of weight ratio to 30/70, oil loss increased to 90%. This may indicate that the presence of byproducts such as lecithin, phosphorus lipids, and sterols in crude omega-3 canola oil improved the anti-seepage capability of CIE products.

TABLE 4
Oil loss content of CIE products.

| | OIL LOSS (v/v) | | | |
|---|---|---|---|---|
| | CIE Palm | CIE Palm | CIE Hydro | CIE fully |
| | stearin/RBD | stearin/Crude | Cotton/Crude | hydro rapeseed |
| Ratio | omega-3 | omega-3 | omega-3 | oil/Crude omega- |
| (w/w) | canola oil | canola oil | canola oil | 3 canola oil |
| 80/20 | 0 | 0 | 0 | 0 |
| 70/30 | 0 | 0 | 0 | 0 |
| 60/40 | 0 | 0 | 0 | 0 |
| 50/50 | 6% | 0 | 0 | 0 |
| 40/60 | 70% | 0 | 0 | 0 |
| 30/70 | 90% | 0 | 0 | 0 |

Table 5 shows basic fatty acid profiles of various oils, and Table 6 shows oil loss content of different oil formulation in a typical diet composition, wherein CIE product 1 is chemical interesterified omega-3 canola oil and fully hydrogenated high erucic rapeseed oil with weight ratio of 40 to 60, and CIE product 2 is chemical interesterified omega-3 canola oil and fully hydrogenated high erucic rapeseed oil with weight ratio of 30 to 70. With the replacement of fish oil with crude omega-3 canola oil, oil loss content decreased from 33.9% to 1.71%. A further improvement of anti-seepage ability of oil mixture was observed using interesterified product to replace hard fat portion in diet. Only 0.42% of oil loss was detected when using interesterified omega-3 canola oil and fully hydrogenated high erucic rapeseed oil with weight of 40 to 60 to replace palm stearin portion in formulation, even though the total saturated fat content is 0.1% lower in diet with interesterified product (#3, Table 6) than that in diet with palm stearin (#2, Table 6). When using interesterified product to replace palm stearin portion in the formulation, with an increase of weight ratio of fully hydrogenated high erucic rapeseed oil to omega-3 canola oil, oil loss content decreased. In formulation of #4, no oil loss was observed. In addition, at the same content of EPA and DHA or saturated fat in diet, the replacement of palm stearin with interesterified product demonstrated very low oil loss which is less than 0.1% (e.g. formulation #5 and #6). All these indicate that the replacement of hard fat with interesterified product in diet for aquafeed feed not only improves anti-seepage ability of oil but also offers a diet with higher content of EPA and DHA.

TABLE 5
Basic fatty acid profiles of oils

| | | Omega-3 | Fish | Canola | Palm |
|---|---|---|---|---|---|
| FAP | Unit | canola oil | oil | oil | Stearin |
| SFAs | g/100 g | 8.43 | 32.85 | 6.63 | 66.78 |
| EPA + DHA | g/100 g | 6.41 | 22.63 | 0 | 0 |
| EPA | g/100 g | 5.69 | 13.52 | 0 | 0 |
| DHA | g/100 g | 0.72 | 9.11 | 0 | 0 |
| DPA | g/100 g | 2.47 | 2.86 | 0 | 0 |
| C18:3:n3 | g/100 g | 5.93 | 1.95 | 9.37 | 0.14 |
| C18:3:n3 + | g/100 g | 8.4 | 4.81 | 9.37 | 0.14 |
| DPA | | | | | |

TABLE 6
Diet formulation and oil loss content.

| Diet composition | Unit | #1 | #2 | #3 | #4 | #5 | #6 |
|---|---|---|---|---|---|---|---|
| Fish oil | % diet | 5.44 | 0 | 0 | 0 | 0 | 0 |
| Omega-3 canola oil | % diet | 0 | 19.2 | 19.2 | 19.2 | 17.34 | 19.02 |
| Canola oil | % diet | 15.56 | 0 | 0 | 0 | 0 | 0 |
| Palm stearin | % diet | 1 | 2.8 | 0 | 0 | 0 | 0 |
| CIE product 1 | % diet | 0 | 0 | 2.8 | 0 | 4.66 | 2.98 |
| CIE product 2 | % diet | 0 | 0 | 0 | 2.8 | 0 | 0 |
| Saturated Fat content | g/100 g | 3.49 | 3.49 | 3.39 | 3.65 | 4.41 | 3.49 |
| EPA + DHA content | g/100 g | 1.23 | 1.23 | 1.30 | 1.28 | 1.23 | 1.30 |
| Oil loss | (%) | 33.9 | 1.71 | 0.42 | 0 | 0 | 0.10 |

### Example 6. Bioavailability

Bioavailability refers to the extent by which a nutrient can be absorbed and transported to systemic circulation or the site of physiological activity. Bioavailability of lipids can be influenced by their chemical and physical properties, such as lipid source, chain length of fatty acids, degree of saturation, and the melting point of fatty acids. Generally, unsaturated fatty acids are absorbed faster than saturated fatty acids. Low chain saturated fatty acids and/or mid chain saturated fatty acids are digested faster than long chain saturated fatty acids (SAFA). A digestible lipid content model supported by multiple experimental results suggests that saturated fatty acids can be incorporated at levels up to approximately 23% of dietary total fatty acids without negatively affecting lipid digestibility. Above this threshold, the lipid digestibility decreases with an increase of saturated fatty acid content in salmonid fish feed. In addition, a significant interaction between saturated fatty acids and unsaturated fatty acids has been observed. This synergistic effect of polyunsaturated fatty acids could improve the digestibility of saturated fatty acids in diet, due to improved emulsifying capacity of unsaturated fatty acids. Fats with higher melting points can show slower digestibility. Saturated fat is an important ingredient in fish feed as too little saturation in fat can result in fat leakage due to low viscosity and impact growth. Therefore, interesterified fats can replace saturated fat with additional benefits of omega-3 fatty acid addition and higher digestibility. Interesterification could improve the digestibility of fat blends.

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present invention. Thus, it should be understood that although the present invention has been specifically disclosed by specific embodiments and optional features, modification and variation of the concepts herein disclosed may be resorted to by those of ordinary skill in the art, and that such modifications and variations are considered to be within the scope of embodiments of the present invention.

### Exemplary Embodiments

The following exemplary embodiments are provided, the numbering of which is not to be construed as designating levels of importance:

Embodiment 1 provides a modified triglyceride comprising:

an omega-3 fatty acid residue; and

a saturated fatty acid residue.

Embodiment 2 provides the modified triglyceride of Embodiment 1, wherein the omega-3 fatty acid residue comprises a (C₈-C₃₀)fatty acid residue comprising 1 to 8 carbon-carbon double bonds.

Embodiment 3 provides the modified triglyceride of any one of Embodiments 1-2, wherein the omega-3 fatty acid residue comprises alpha-linolenic acid (ALA), eicosapentaenoic acid (EPA), docosapentaenoic acid (DPA), and docosahexaenoic acid (DHA).

Embodiment 4 provides the modified triglyceride of any one of Embodiments 1-3, wherein the saturated fatty acid residue comprises a (C₈-C₃₀)fatty acid residue.

Embodiment 5 provides the modified triglyceride of any one of Embodiments 1-4, wherein the saturated fatty acid residue comprises (C₁₆-C₂₂)fatty acid residue.

Embodiment 6 provides the modified triglyceride of any one of Embodiments 1-5, wherein the modified triglyceride is substantially free of free fatty acids.

Embodiment 7 provides a food comprising the modified triglyceride of any one of Embodiments 1-6.

Embodiment 8 provides the food of Embodiment 7 wherein the food is a fish feed.

Embodiment 9 provides the food of Embodiment 8, wherein the fish feed is a salmon feed.

Embodiment 10 provides the food of any one of Embodiments 7-9, wherein the modified triglyceride of any one of Embodiments 1-6 is about 0.1 wt % to about 100 wt % of the food.

Embodiment 11 provides the food of any one of Embodiments 7-10, wherein the modified triglyceride of any one of Embodiments 1-6 is about 2.5 wt % to about 20 wt % of the food.

Embodiment 12 provides a method of forming the food of any one of Embodiments 7-11, comprising adding the modified triglyceride to an edible composition to form the food.

Embodiment 13 provides a structured fat blend comprising: the modified triglyceride of any one of Embodiments 1-6.

Embodiment 14 provides the structured fat blend of Embodiment 13, further comprising a triglyceride comprising at least one saturated fatty acid residue.

Embodiment 15 provides the structured fat blend of any one of Embodiments 13-14, wherein the modified triglyceride is about 1 wt % to about 99 wt % of the blend.

Embodiment 16 provides the structured fat blend of any one of Embodiments 13-15, wherein the modified triglyceride is about 20 wt % to about 80 wt % of the blend.

Embodiment 17 provides the structured fat blend of any one of Embodiments 14-16, wherein the triglyceride comprising at least one saturated fatty acid residue is about 1 wt % to about 99 wt % of the blend.

Embodiment 18 provides the structured fat blend of any one of Embodiments 14-17, wherein the triglyceride comprising at least one saturated fatty acid residue is about 20 wt % to about 80 wt % of the blend.

Embodiment 19 provides the structured fat blend of any one of Embodiments 13-18, wherein the structured fat blend is an interesterified product of a starting material triglyceride comprising an omega-3 fatty acid residue and a highly saturated triglyceride.

Embodiment 20 provides the structured fat blend of any one of Embodiments 13-19, wherein the structured fat blend is an interesterified product of an omega-3 canola oil and palm stearin, fully hydrogenated cotton seed oil, fully hydrogenated high erucic rapeseed oil, fully hydrogenated soybean oil, or a combination thereof.

Embodiment 21 provides the structured fat blend of any one of Embodiments 13-20, wherein the structured fat blend has a lower melting point than a blend of a starting material triglyceride comprising the omega-3 fatty acid residue and a highly saturated triglyceride used to form the structured fat blend.

Embodiment 22 provides the structured fat blend of any one of Embodiments 13-21, wherein the structured fat blend has a lower solid fat content (SFC) than a blend of a starting material triglyceride comprising the omega-3 fatty acid residue and a highly saturated triglyceride used to form the structured fat blend.

Embodiment 23 provides the structured fat blend of any one of Embodiments 13-22, wherein the structured fat blend, as compared to a blend of a starting material triglyceride comprising the omega-3 fatty acid residue and a highly saturated triglyceride used to form the structured fat blend, has about 55% less to about 1 wt % less trisaturated (S3) triglyceride content.

Embodiment 24 provides the structured fat blend of any one of Embodiments 13-23, wherein the structured fat blend, as compared to a blend of a starting material triglyceride comprising the omega-3 fatty acid residue and a highly saturated triglyceride used to form the structured fat blend, has about 2 wt % to 35 wt % less trisaturated (S3) triglyceride content.

Embodiment 25 provides the structured fat blend of any one of Embodiments 13-24, wherein the structured fat blend, as compared to a blend of a starting material triglyceride comprising the omega-3 fatty acid residue and a highly saturated triglyceride used to form the structured fat blend, has about 20 wt % less to about 70 wt % more disaturated-monounsaturated (S2U) triglyceride content.

Embodiment 26 provides the structured fat blend of any one of Embodiments 13-25, wherein the structured fat blend, as compared to a blend of a starting material triglyceride comprising the omega-3 fatty acid residue and a highly saturated triglyceride used to form the structured fat blend, has about 1 wt % to about 50 wt % more disaturated-monounsaturated (S2U) triglyceride content.

Embodiment 27 provides the structured fat blend of any one of Embodiments 13-26, wherein the structured fat blend, as compared to a blend of a starting material triglyceride comprising the omega-3 fatty acid residue and a highly saturated triglyceride used to form the structured fat blend, has about 25 wt % less to about 70 wt % more monsaturated-diunsaturated (SU2) triglyceride content.

Embodiment 28 provides the structured fat blend of any one of Embodiments 13-27, wherein the structured fat blend, as compared to a blend of a starting material triglyceride comprising the omega-3 fatty acid residue and a highly saturated triglyceride used to form the structured fat blend, has about 5% less to about 20 wt % more monsaturated-diunsaturated (SU2) triglyceride content.

Embodiment 29 provides the structured fat blend of any one of Embodiments 13-28, wherein the structured fat blend, as compared to a blend of a starting material triglyceride comprising the omega-3 fatty acid residue and a highly saturated triglyceride used to form the structured fat blend, has about 55 wt % less to about 1 wt % less triunsaturated (U3) triglyceride content.

Embodiment 30 provides the structured fat blend of any one of Embodiments 13-29, wherein the structured fat blend, as compared to a blend of a starting material triglyceride comprising the omega-3 fatty acid residue and a highly saturated triglyceride used to form the structured fat blend, has about 2 wt % to about 25 wt % less triunsaturated (U3) triglyceride content.

Embodiment 31 provides the structured fat blend of any one of Embodiments 13-30, wherein trisaturated (S3) triglycerides are about 5 wt % to about 95 wt % of the structured fat blend.

Embodiment 32 provides the structured fat blend of any one of Embodiments 13-31, wherein trisaturated (S3) triglycerides are about 6 wt % to about 50 wt % of the structured fat blend.

Embodiment 33 provides the structured fat blend of any one of Embodiments 13-32, wherein disaturated-monounsaturated (S2U) triglycerides are about 1 wt % to about 70 wt % of the structured fat blend.

Embodiment 34 provides the structured fat blend of any one of Embodiments 13-33, wherein disaturated-monounsaturated (S2U) triglycerides are about 19 wt % to about 51 wt % of the structured fat blend.

Embodiment 35 provides the structured fat blend of any one of Embodiments 13-34, wherein monsaturated-diunsaturated (SU2) triglycerides are about 1 wt % to about 70 wt % of the structured fat blend.

Embodiment 36 provides the structured fat blend of any one of Embodiments 13-35, wherein monsaturated-diunsaturated (SU2) triglycerides are about 5 wt % to about 47 wt % of the structured fat blend.

Embodiment 37 provides the structured fat blend of any one of Embodiments 13-36, wherein triunsaturated (U3) triglycerides are about 1 wt % to about 60 wt % of the structured fat blend.

Embodiment 38 provides the structured fat blend of any one of Embodiments 13-37, wherein triunsaturated (U3) triglycerides are about 1 wt % to about 39 wt % of the structured fat blend.

Embodiment 39 provides a food comprising the structured fat blend of any one of Embodiments 13-38.

Embodiment 40 provides the food of Embodiment 39, wherein the food comprises a blend of an edible composition and the structured fat blend.

Embodiment 41 provides the food of any one of Embodiments 39-40, wherein the food is a fish feed.

Embodiment 42 provides the food of Embodiment 41, further comprising fish oil.

Embodiment 43 provides the food of Embodiment 42, wherein the fish feed is a salmon feed.

Embodiment 44 provides the food of any one of Embodiments 41-43, wherein when placed in water the food experiences less leakage of the omega-3 fatty acid residue from the food than the same food having the modified triglyceride replaced with the starting material triglyceride comprising the omega-3 fatty acid used to form the structured fat blend.

Embodiment 45 provides the food of any one of Embodiments 39-44, wherein the structured fat blend is about 0.1 wt % to about 100 wt % of the food.

Embodiment 46 provides the food of any one of Embodiments 39-45, wherein the structured fat blend of is about 2.5 wt % to about 20 wt % of the food.

Embodiment 47 provides a fish feed comprising a structured fat blend of any one of Embodiments 13-38 that is an interesterified product of omega-3 canola oil and palm stearin, fully hydrogenated cotton seed oil, fully hydrogenated high erucic rapeseed oil, fully hydrogenated soybean oil, or a combination thereof.

Embodiment 48 provides a method of forming the food of any one of Embodiments 39-47, comprising adding the structured fat blend to an edible composition to form the food.

Embodiment 49 provides a method of making the modified triglyceride of any one of Embodiments 1-6 or the structured fat blend of any one of Embodiments 13-38, the method comprising:

reacting a fatty acid source comprising at least one of an omega-3 free fatty acid or a salt thereof and an ester of an omega-3 fatty acid with a highly saturated triglyceride to form the triglyceride comprising the omega-3 fatty acid residue and the saturated fatty acid residue.

Embodiment 50 provides the method of claim 49, wherein the highly saturated triglyceride comprises a fully hydrogenated triglyceride.

Embodiment 51 provides the method of any one of Embodiments 49-50, wherein the highly saturated triglyceride comprises palm stearin, fully hydrogenated cotton seed oil, fully hydrogenated high erucic rapeseed oil, fully hydrogenated soybean oil, or a combination thereof.

Embodiment 52 provides the method of any one of Embodiments 49-51, wherein the fatty acid source comprises the omega-3 free fatty acid.

Embodiment 53 provides the method of Embodiment 52, further comprising separating a non-omega 3-containing free fatty acid product of the reaction from the triglyceride comprising the omega-3 fatty acid residue and the saturated fatty acid residue.

Embodiment 54 provides the method of Embodiment 53, wherein the separated free fatty acid product corresponds to a fatty acid residue on the highly saturated triglyceride that was replaced by the omega-3 free fatty acid.

Embodiment 55 provides the method of Embodiment 49, wherein the fatty acid source is the ester of the omega-3 fatty acid, wherein the reaction is an interesterification reaction.

Embodiment 56 provides the method of Embodiment 55, wherein the interesterification reaction is a chemical esterification reaction.

Embodiment 57 provides the method of any one of Embodiments 55-56, wherein the interesterification is an enzymatic interesterification.

Embodiment 58 provides the method of Embodiment 57, wherein the enzymatic interesterification comprises interesterification facilitated by one or more enzymes.

Embodiment 59 provides the method of any one of Embodiments 55-58, wherein the ester of the omega-3 fatty acid is a (C₁-C₂₀)hydrocarbyl ester.

Embodiment 60 provides the method of any one of Embodiments 55-59, wherein the ester of the omega-3 fatty acid is a (C₁-C₅)alkyl ester

Embodiment 61 provides the method of any one of Embodiments 55-60, wherein the ester of the omega-3 fatty acid is a triglyceride comprising a residue of the omega-3 fatty acid.

Embodiment 62 provides the method of Embodiment 61, wherein the triglyceride comprising the residue of the omega-3 fatty acid is algal oil, omega-3 canola oil, a plant-based oil including omega-3 fatty acid residues, a marine-based oil, a non-marine-based oil, or a combination thereof.

Embodiment 63 provides the method of any one of Embodiments 61-62, wherein the triglyceride comprising the residue of the omega-3 fatty acid is omega-3 canola oil.

Embodiment 64 provides the method of any one of Embodiments 61-63, wherein the triglyceride comprising the residue of the omega-3 fatty acid is crude omega-3 canola oil.

Embodiment 65 provides the method of any one of Embodiments 61-64, wherein the triglyceride comprising the residue of the omega-3 fatty acid is a crude triglyceride.

Embodiment 66 provides the method of Embodiment 65, wherein the crude triglyceride comprising the residue of the omega-3 fatty acid comprises a sterol, a tocopherol, lecithin, a phosphorus lipid, or a combination thereof.

Embodiment 67 provides a method of making a fish feed, comprising performing the method of any one of Embodiments 49-66 to form the triglyceride comprising the omega-3 fatty acid residue and the saturated fatty acid residue, further comprising adding the triglyceride comprising the omega-3 fatty acid residue and the saturated fatty acid residue to an edible composition to form the fish feed.

Embodiment 68 provides the modified triglyceride, food, structured fat blend, or method of any one or any combination of Embodiments 1-67 optionally configured such that all elements or options recited are available to use or select from.

1. A modified triglyceride comprising:
an omega-3 fatty acid residue; and
a saturated fatty acid residue.

2. The modified triglyceride of claim 1, wherein the omega-3 fatty acid residue comprises alpha-linolenic acid (ALA), eicosapentaenoic acid (EPA), docosapentaenoic acid (DPA), and docosahexaenoic acid (DHA).

3. The modified triglyceride of claim 1, wherein the modified triglyceride is substantially free of free fatty acids.

4. A food comprising the modified triglyceride of claim 1.

5. The food of claim 4 wherein the food is a fish feed.

6. A method of forming the food of claim 4, comprising adding the modified triglyceride to an edible composition to form the food.

7. A structured fat blend comprising:
the modified triglyceride of claim 1.

8. The structured fat blend of claim 7, further comprising a triglyceride comprising at least one saturated fatty acid residue.

9. The structured fat blend of claim 7, wherein the structured fat blend is an interesterified product of a starting material triglyceride comprising an omega-3 fatty acid residue and a highly saturated triglyceride.

10. The structured fat blend of claim 7, wherein the structured fat blend is an interesterified product of an omega-3 canola oil and palm stearin, fully hydrogenated cotton seed oil, fully hydrogenated high erucic rapeseed oil, fully hydrogenated soybean oil, or a combination thereof.

11. The structured fat blend of claim 7, wherein the structured fat blend has a lower melting point than a blend of a starting material triglyceride comprising the omega-3 fatty acid residue and a highly saturated triglyceride used to form the structured fat blend.

12. The structured fat blend of claim 7, wherein the structured fat blend has a lower solid fat content (SFC) than a blend of a starting material triglyceride comprising the omega-3 fatty acid residue and a highly saturated triglyceride used to form the structured fat blend.

13. A food comprising the structured fat blend of claim 7.

14. The food of claim 13, wherein the food is a fish feed.

15. The food of claim 14, wherein when placed in water the food experiences less leakage of the omega-3 fatty acid residue from the food than the same food having the modified triglyceride replaced with the starting material triglyceride comprising the omega-3 fatty acid used to form the structured fat blend.

16. A fish feed comprising a structured fat blend of claim 7 that is an interesterified product of omega-3 canola oil and palm stearin, fully hydrogenated cotton seed oil, fully hydrogenated high erucic rapeseed oil, fully hydrogenated soybean oil, or a combination thereof.

17. A method of forming the food of claim 13, comprising adding the structured fat blend to an edible composition to form the food.

18. A method of making the modified triglyceride of claim 1, the method comprising:
reacting a fatty acid source comprising at least one of an omega-3 free fatty acid or a salt thereof and an ester of an omega-3 fatty acid with a highly saturated triglyceride to form the triglyceride comprising the omega-3 fatty acid residue and the saturated fatty acid residue.

19. A method of making the structured fat blend of claim 7, the method comprising:
reacting a fatty acid source comprising at least one of an omega-3 free fatty acid or a salt thereof and an ester of an omega-3 fatty acid with a highly saturated triglyceride to form the triglyceride comprising the omega-3 fatty acid residue and the saturated fatty acid residue.

20. A method of making a fish feed, comprising performing the method of claim 18 to form the triglyceride comprising the omega-3 fatty acid residue and the saturated fatty acid residue, further comprising adding the triglyceride comprising the omega-3 fatty acid residue and the saturated fatty acid residue to an edible composition to form the fish feed.
